(19) United States
(12) Patent Application Publication
(10) Pub. No.: US 2007/0043121 A1
(43) Pub. Date: Feb. 22, 2007

(54) Discovery of novel soluble crystalline anesthetics

(76) Inventors: Milton L. Brown, Charlottesville, VA (US); Hilary A. Schenk, York, PA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/573,910

(22) PCT Filed: Sep. 29, 2004
(86) PCT No.: PCT/US04/31940

§ 371 Date: Mar. 29, 2006

Related U.S. Application Data
(60) Provisional application No. 60/507,196, filed on Sep. 29, 2003.

(51) Int. Cl. A61K 31/165; C07C 235/56

(52) U.S. Cl. 514/622; 564/133; 564/170

(57) ABSTRACT

The invention discloses compounds derived from propofal which have greater aqueous solubility than propofal and are useful as anesthetic agents. The invention further discloses methods of preparing compounds of the invention. The invention also discloses methods of inducing anesthesia in a subject by administering compounds of the invention to the subject.

### CROSS REFERENCE TO RELATED APPLICATIONS

The present application claims priority under 35 U.S.C. §119(e) to U.S. Provisional Application No. 60/507,196 filed on Sep. 29, 2003.

### FIELD OF THE INVENTION

The invention relates to compounds derived from propofal which have greater aqueous solubility than propofal and are useful as anesthetic agents. The invention further relates to methods of preparing compounds derived from propofal. The invention also discloses methods of inducing anesthesia in a subject by administering compounds of the invention to the subject.

### BACKGROUND

Propofal, a simple formulation of 2,6-diisopropylphenol is an effective intravenous anesthetic, and is used for induction and maintenance of general anesthesia. Propofal is also known as 2,6-bis(1-methylethyl)phenol. Propofal administration can lower blood pressure due to a decrease in systemic vascular resistance, cardiac contractility (Hirota, K., Masuda, A., Ito, Y. Anesthesia & Analgesia. 1999, 89:225-9) and preload. Propofal can also produce profound respiratory depression when used to induce anesthesia and conscious sedation and significantly inhibits hypoxic ventilatory drive. This compound, when administered by continuous infusion or by intermittent bolus doses, has found increased post-operative use for its quick anesthetic onset and reversibility.

Unfortunately, unmodified propofal has significant limitations in its use due to poor aqueous solubility (propofal is an oil and thus is very water insoluble) and must be formulated as an oil: water emulsion. Propofal has demonstrated sodium activity by inhibiting 3H-batrachotoxin at 26 micromolar. The present invention is directed to a propofal derivative molecule that incorporates propofal into its core but makes the molecule more water soluble and allows for a crystalline (solid) preparation. The general structure for propofal (I) is:

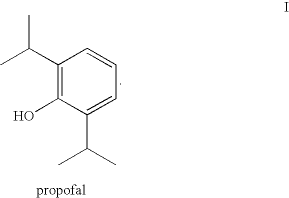

There is a long felt need in the art for the development of new derivatives of propofal and methods of preparing and using such compounds, especially compounds which have anesthetic activity. The present invention satisfies these needs.

### SUMMARY OF THE INVENTION

The present invention, as described in the disclosure provided herein, is based on the discovery that derivatives of propofal have crystalline properties and have greater aqueous solubility than propofal. The summary, as well as the following detailed description of preferred embodiments of the invention, will be better understood when read in conjunction with the chemical structures and formulas provided herein. For the purpose of illustrating the invention, there are shown by chemical structures and formulas embodiments which are presently preferred. It should be understood, however, that the invention is not limited to the precise arrangements and instrumentalities provided herein.

In one aspect, compounds of the present invention are derivatives of propofal (structure I).

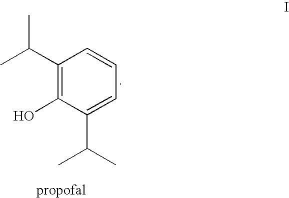

In another aspect, the invention is directed to propofal derivative compounds having general structures selected from the group consisting of structures II, III, IV, and V as follows:

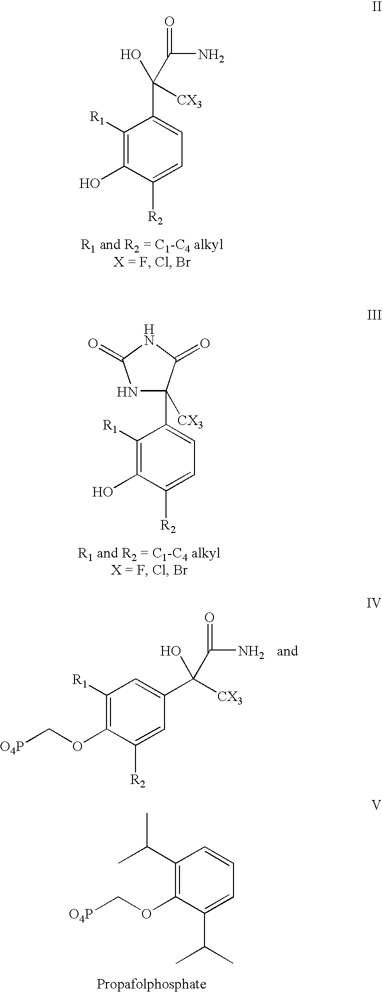

In one aspect, a propofal (I) derivative compound of the invention has the general structural formula (VI) as follows:

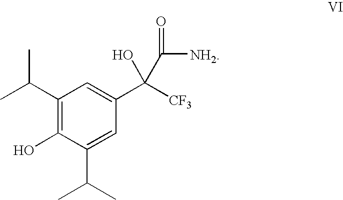

In one aspect, compounds of the invention can be prepared in crystalline form and are more water soluble than propofal (I). In another aspect, compounds of the invention are useful as anesthetic agents. In another aspect, compounds of the invention are prepared according to Scheme I, as provided herein.

### DETAILED DESCRIPTION OF THE INVENTION

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, an anesthetic agent is one which can be used to induce anesthesia.

A “compound,” as used herein, refers to any type of substance or agent that is commonly considered a drug, or a candidate for use as a drug, as well as combinations and mixtures of the above.

A “derivative compound,” as used herein, refers to a compound derived from propofal, said derivative compound having the basic propofal structural formula (i.e., structural formula I) which has been modified according to the description provided herein. A “derivative compound” as used herein includes compounds which incorporate the basic structural formula of propofal (I) as described herein.

An “effective amount” or “therapeutically effective amount” of a compound is that amount of compound which is sufficient to provide a beneficial or selected effect to the subject to which the compound is administered. For example, an effective amount of a propofal derivative is an amount of the compound sufficient to reduce the incidence of seizures in a patient receiving the dose amount.

As used herein, a “functional” compound or molecule is a compound molecule in a form in which it exhibits a property by which it is characterized. A functional propofal derivative is one which has the biological properties of propofal.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

The MAC value is the minimum alveolar concentration of anesthetic at 1 atm that produces immobility in 50% of the subjects.

The term, “parenteral” means not through the alimentary canal but by some other route such as subcutaneous, intramuscular, intraspinal, or intravenous.

As used herein, the term “pharmaceutically-acceptable carrier” means a chemical composition with which an appropriate compound or derivative can be combined and which, following the combination, can be used to administer the appropriate compound to a subject. The term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “physiologically acceptable” ester or salt means an ester or salt form of the active ingredient which is compatible with any other ingredients of the pharmaceutical composition, which is not deleterious to the subject to which the composition is to be administered.

As used herein, the term “purified” and like terms relate to the isolation of a molecule or compound in a form that is substantially free of contaminants normally associated with the molecule or compound in a native or natural environment.

As used herein, the term “treating” includes prophylaxis of the specific disorder or condition, or alleviation of the symptoms associated with a specific disorder or condition and/or preventing or eliminating said symptoms.

A “subject” which is administered a compound of the invention is a mammal, including a human. Non-human animals subject to administration of a compound of the invention include, but are not limited to, primates, cats, dogs, horses, cows, goats, and sheep.

The term “substantially pure” describes a compound, e.g., a propofal derivative which has been separated from components which naturally accompany it during synthesis. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method.

“Synthesis of a propofal derivative,” as used herein refers to the formation or production of a derivative of the basic propofal structural formula (i.e., structural formula I).

Chemical Groups

As used herein the term “aryl” refers to a mono- or bicyclic carbocyclic ring system having one or two aromatic rings including, but not limited to, phenyl, benzyl, naphthyl, tetrahydronaphthyl, indanyl, indenyl, and the like. “Optionally substituted aryl” includes aryl compounds having from zero to four substituents, and “substituted aryl” includes aryl compounds having one to three substituents. The term (C₅-C₈ alkyl)aryl refers to any aryl group which is attached to the parent moiety via the alkyl group.

The term “bicyclic” represents either an unsaturated or saturated stable 7- to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

The term “C₁-Cₙ alkyl” wherein n is an integer, as used herein, represents a branched or linear alkyl group having from one to the specified number of carbon atoms. Typically, C₁-C₆ alkyl groups include, but are not limited to, methyl, ethyl, n-propyl, iso-propyl, butyl, iso-butyl, sec-butyl, tert-butyl, pentyl, hexyl, and the like.

The term “C₂-Cₙ alkenyl” wherein n is an integer, as used herein, represents an olefinically unsaturated branched or linear group having from 2 to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer refers to an unsaturated branched or linear group having from 2 to the specified number of carbon atoms and at least one triple bond. Examples of such groups include, but are not limited to, 1-propynyl, 2-propynyl, 1-butynyl, 2-butynyl, 1-pentynyl, and the like.

The term “C₃-Cₙ cycloalkyl” wherein n is an integer refers to cyclic non-aryl group, for example C₃-C₈ cycloalkyl, represents cyclopropyl, cyclobutyl, cyclopentyl, cyclohexyl, cycloheptyl, and cyclooctyl.

As used herein, the term “halo” means Cl, Br, F, and I. Especially preferred halogens include Cl, Br, and F. The term “haloalkyl” as used herein refers to a C₁-Cₙ alkyl radical bearing at least one halogen substituent, for example, chloromethyl, fluoroethyl or trifluoromethyl and the like.

As used herein the term “heteroaryl” refers to a mono- or bicyclic carbocyclic ring system having one or two aromatic rings containing from one to three heteroatoms and includes, but is not limited to, furyl, thienyl, pyridyl and the like.

The term “heterocyclic group” refers to a mono- or bicyclic carbocyclic ring system containing from one to three heteroatoms wherein the heteroatoms are selected from the group consisting of oxygen, sulfur, and nitrogen.

The term “lower alkyl” as used herein refers to branched or straight chain alkyl groups comprising one to eight carbon atoms, including methyl, ethyl, propyl, isopropyl, n-butyl, t-butyl, neopentyl, and the like.

As used herein, the term “optionally substituted” refers to from zero to four substituents, wherein the substituents are each independently selected. Each of the independently selected substituents may be the same or different than other substituents.

Compounds of the present invention that have one or more asymmetric carbon atoms may exist as the optically pure enantiomers, or optically pure diastereomers, as well as mixtures of enantiomers, mixtures of diastereomers, and racemic mixtures of such stereoisomers. The present invention includes within its scope all such isomers and mixtures thereof.

Examples of The Invention

In accordance with one embodiment, the novel propofal derivatives of the present invention have the general structure of formulas II, III, IV, V, and VI as follows:

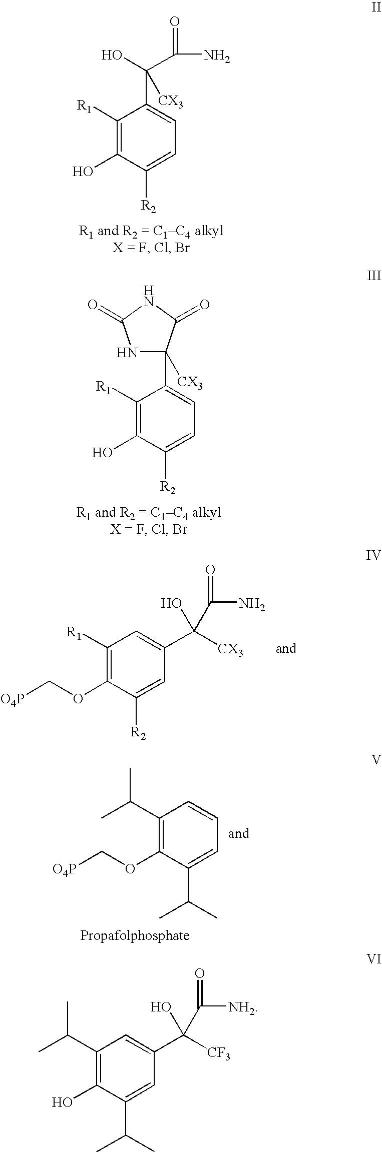

In one embodiment, R₁ and R₂ are independently selected from the group consisting of C₁-C₄ alkyl and X is Br, Cl, or F. In another embodiment, R₁ and R₂ are each isopropyl and X is halo, and in a further embodiment, R₁ and R₂ are each isopropyl and X is F. In a further embodiment of the invention, a compound having anesthetic activity is provided wherein R₁ is C₁-C₄ alkyl, X is halo, and R₂ is selected from the group consisting of hydroxy, alkoxy and −O(CH₂)ₙPO₄, and n is an integer ranging from 1-4. In yet a further embodiment, R₁ is isopropyl, X is Cl or F and R₂ is hydroxy or −O(CH₂)ₙPO₄, wherein n is an integer ranging from 1-4. In yet another embodiment, R₁ is isopropyl, X is F and R₂ is hydroxy.

In accordance with one embodiment, the propofal derivatives of the present invention can be formulated as pharmaceutical compositions by combining the compounds with one or more pharmaceutically acceptable carriers, fillers, solubilizing agents and stabilizers known to those skilled in the art. Such pharmaceutical compositions can be utilized as analgesics, sedatives, anesthetics or as anticonvulsants.

Pharmaceutical compositions comprising the propofal derivatives of the present invention are administered to an individual in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means, with oral and intravenous routes being preferred. When administered orally, the compounds can be administered as a liquid solution, powder (lyophilized or otherwise), tablet, capsule, or lozenge. Furthermore, oral formulations may include one or more of the present compounds in combination with one or more conventional pharmaceutical additive or excipients that are typically used in the preparation of tablets, capsules, lozenges, and other orally administrable forms. When administered as an intravenous solution, the derivatives of the present invention can be admixed with conventional IV solutions to form injectable aqueous or oily suspensions or solutions.

In accordance with one embodiment, a propofal derivative of the present invention is combined with other known anesthetic agents or other propofal derivatives to enhance the performance of such compounds and decrease the incidence of negative side effects. For example, compositions according to the present invention may comprise a propofal derivative and a phencyclidine type general anesthetic such as ketamine or tiletamine and their pharmaceutically acceptable salts, as well as selegiline or one of its pharmaceutically acceptable salts, combined in a single pharmaceutical composition for simultaneous administration, or presented separately for administration in close succession. In the latter case, selegiline has the role of pre-anesthetic or restraining agent. Tiletamine is 2-(ethylamino)-2-(2-thienyl)cyclohexanone. Ketamine is (±)-2-(2-chlorophenyl)-2-methyl-aminocyclohexanone. Selegiline is (−)-N, alpha-dimethyl-N-(2-propynyl)phenethylamine.

The invention relates to administration of an identified compound in a pharmaceutical composition to practice the methods of the invention, the composition comprising the compound or an appropriate derivative or fragment of the compound and a pharmaceutically-acceptable carrier. As used herein, the term “pharmaceutically-acceptable carrier” means a chemical composition with which an appropriate compound of the invention may be combined and which, following the combination, can be used to administer the appropriate compound to a subject.

In one embodiment, the pharmaceutical compositions useful for practicing the invention may be administered to deliver a dose of between 1 ng/kg/day and 100 mg/kg/day.

Other pharmaceutically acceptable carriers which are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., N.J.).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Different methods and formulations are known in the art for administration of anesthetics. For example, see U.S. Pat. Nos. 6,423,338, 6,669,908, 6,680,331, and 6,790,855, the entireties of which are incorporated herein by reference.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer, for example, a propofal derivative according to the methods of the invention. The method should not be construed to be limited to the general propofal structure, but should be construed to include other derivatives thereof.

The active ingredient may be present in the pharmaceutical composition in the form of a physiologically acceptable ester or salt, such as in combination with a physiologically acceptable cation or anion, as is well known in the art.

Compounds which are identified or prepared using any of the methods described herein may be formulated and administered to a mammal as described herein Methods for identifying compounds with anesthetic activity are known in the art.

The invention also includes a kit comprising a composition of the invention and an instructional material which describes administering the composition to a cell or to a tissue of a mammal. In another embodiment, this kit comprises a (preferably sterile) solvent suitable for dissolving or suspending the composition of the invention prior to administering the compound to the mammal.

The present invention also provides a pharmaceutical pack or kit comprising one or more containers containing one or more of the propofal derivatives of the present invention. In accordance with one embodiment, a kit is provided for anesthetizing a subject. In one aspect, the subject is a human. In this embodiment, the kit may comprise one or more anesthetic agents of the present invention and as well as other known anesthetic agents and pre-anesthetic or restraining agents. These pharmaceuticals can be packaged in a variety of containers, e.g., vials, tubes, microtiter well plates, bottles, and the like. Preferably, the kits will also include instructional materials.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains a compound of the invention or a composition comprising a compound of the invention, or be shipped together with a container which contains the peptide. Alternatively, the instructional material may be shipped separately from the container with the intention that the instructional material and the compound be used cooperatively by the recipient.

In one embodiment, a composition comprising a propofal derivative of the present invention is used as a general anesthesia in mammalian subjects, including both human and domesticated animals. More particularly, compositions comprising the present propofal derivative are administered either orally or parenterally to a mammalian species to induce anesthesia. When administered orally, the compounds are administered as a liquid solution, powder, tablet, capsule, or lozenge. The compounds can be used in combination with one or more conventional pharmaceutical additive or excipients used in the preparation of tablets, capsules, lozenges and other orally administrable forms. When administered parenterally, and more preferably by intravenous injection, the derivatives of the present invention can be admixed with saline solutions and/or conventional IV solutions. Other administration methods may be used and are described herein or are known to those of skill in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, and mammals, including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to a subject, preferably a human, will vary depending upon any number of factors, including but not limited to, the type of subject, the type of surgery or procedure being performed on the subject, the disease state being treated, the age of the subject and the route of administration.

A compound of the invention can be administered to a subject as frequently as several times per hour, or it may be administered more or less frequently. The frequency of the dose will be readily apparent to the skilled artisan and will depend upon any number of factors, such as, but not limited to, the route of administration, the severity of the disease being treated, the type and age of the subject, and the type of surgery or procedure being performed on the subject, etc.

In accordance with one embodiment, a method is provided for inducing anesthesia in a human patient. The method comprises the steps of administering to the patient a composition comprising a compound represented by a formula which is a derivative of formula I. In one aspect, a compound of the invention is selected from the group consisting of formulas II, III, IV, V, and VI, or derivatives thereof.

In one embodiment of the invention, a compound having anesthetic activity is provided wherein the compound has a general structure selected from the group consisting of formulas II, III, and IV, wherein R₁ is C₁-C₄ alkyl, X is halo, and R₂ is selected from the group consisting of hydroxy, alkoxy and —O(CH₂)ₙPO₄, and n is an integer ranging from 1-4. In one embodiment, R₁ is isopropyl, X is Cl or F and R₂ is hydroxy or —O(CH₂)ₙPO₄ wherein n is an integer ranging from 1-4. In another embodiment R₁ is isopropyl, X is F and R₂ is hydroxy.

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

Compounds of the invention may be prepared according to the synthetic schemes provided herein.

### EXAMPLE

### Synthesis of Propofal Derivatives

The following synthetic scheme demonstrates a method of preparing compounds of the invention. Starting components, reagents, conditions, and intermediate steps and compounds are provided in Scheme I and indicated by compounds 1 to 17.

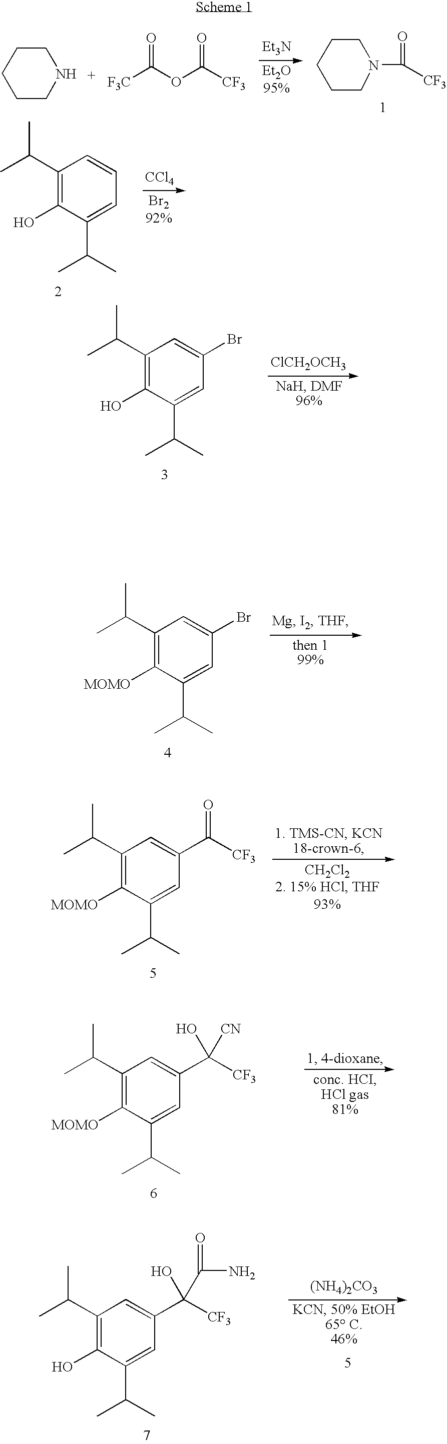

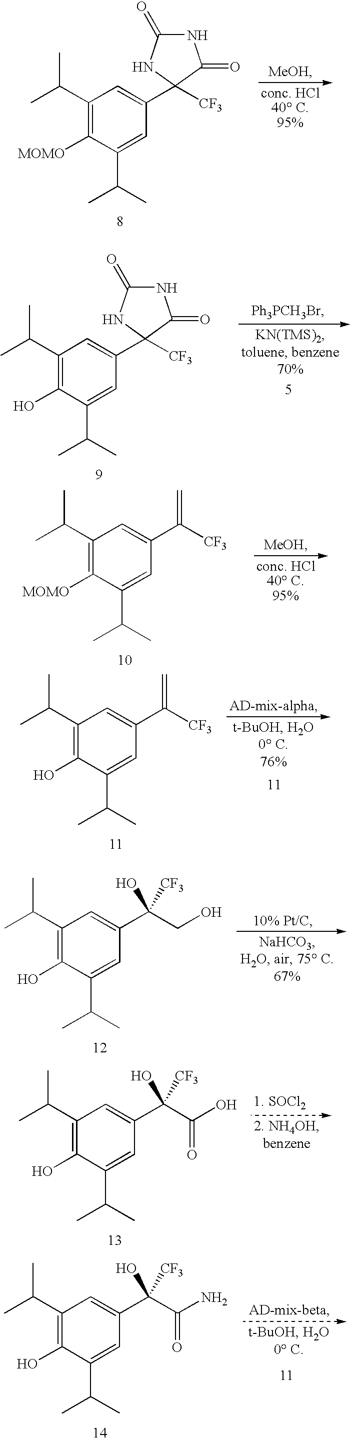

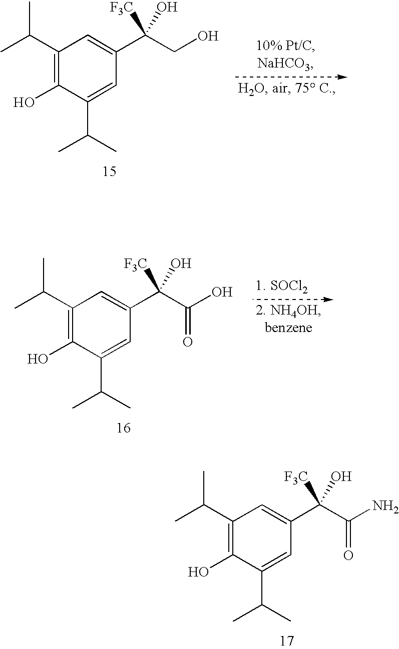

What is claimed is:

1. A compound having the general structural formula of propofal (I) 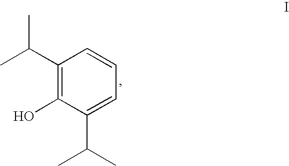
wherein said compound is a derivative of propofal (I) and has greater aqueous solubility than propofal (I), further wherein said compound is an anesthetic agent.

2. A compound of claim 1 having a general structure selected from the group consisting of structures II, III, IV, and V, or derivatives thereof:

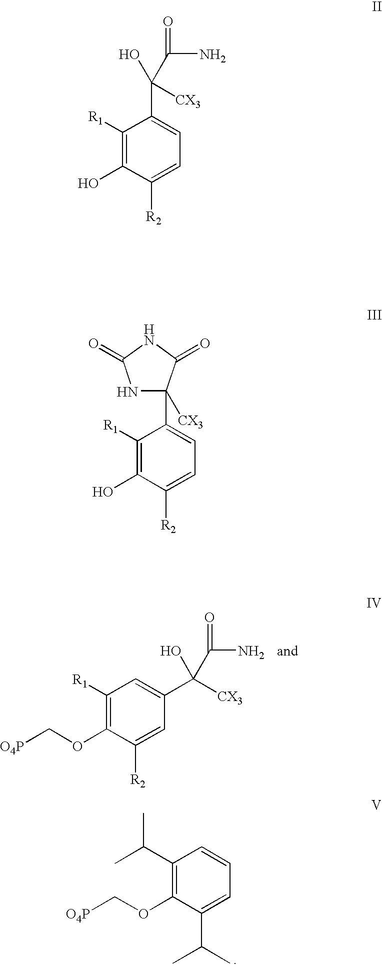

3. A compound of claim 2, wherein R₁ and R₂ are independently selected from the group consisting of isopropyl, C₁-C₄ alkyl, hydroxy, alkoxy and —O(CH₂)ₙPO₄, n is an integer ranging from 1-4, and X is halo.

4. A compound of claim 2, wherein R₁ is independently selected from the group consisting of C₁-C₄ alkyl, X is halo, and R₂ is independently selected from the group consisting of hydroxy, alkoxy and —O(CH₂)ₙPO₄, and n is an integer ranging from 1-4.

5. A compound of claim 2, wherein R₁ and R₂ are independently selected from the group consisting of C₁-C₄ alkyl and X is halo.

6. A compound of claim 5, wherein X is F.

7. A compound of claim 2, wherein R₁ and R₂ are each isopropyl and X is halo.

8. A compound of claim 7, wherein X is F.

9. A compound of claim 2, wherein R₁ is isopropyl, X is halo, and R₂ is hydroxy or —O (CH₂)ₙPO₄, and n is an integer ranging from 1-4.

10. A compound of claim 9, wherein X is F and R₂ is hydroxy.

11. A compound of claim 9, wherein X is Cl or F.

12. A compound of claim 1 having the general structure

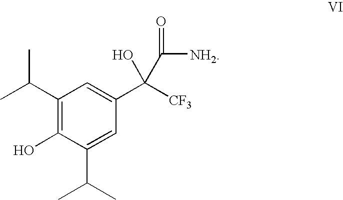

13. A compound according to claims 1, 2, 3, 4, 5, or 12 wherein said compound is crystalline in form.

14. A pharmaceutical composition comprising at least one compound of claims 1, 2, 3, 4, 5, or 12 and a pharmaceutically acceptable carrier.

15. A method of inducing anesthesia in a subject in need thereof, said method comprising administering to said subject an effective amount of a composition comprising at least one compound according to claims 1, 2, 3, 4, 5, or 12, or pharmaceutically acceptable salts thereof.

16. The method of claim 15, wherein said composition is administered intravenously.

17. The method of claim 15, wherein said subject is a human.

18. A kit for inducing anesthesia in a subject in need of anesthesia, said kit comprising a pharmaceutical composition of claim 14, an applicator, and an instructional material for the use thereof.

19. A method of preparing an anesthetic compound derived from propofal (I), said method comprising preparing said compound derived from propofal according to scheme I: 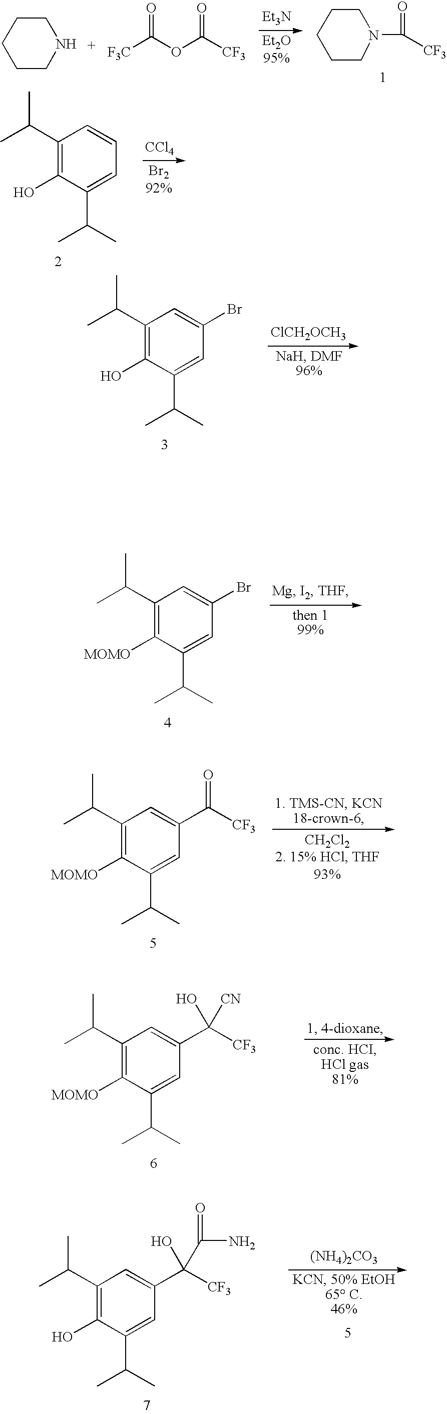 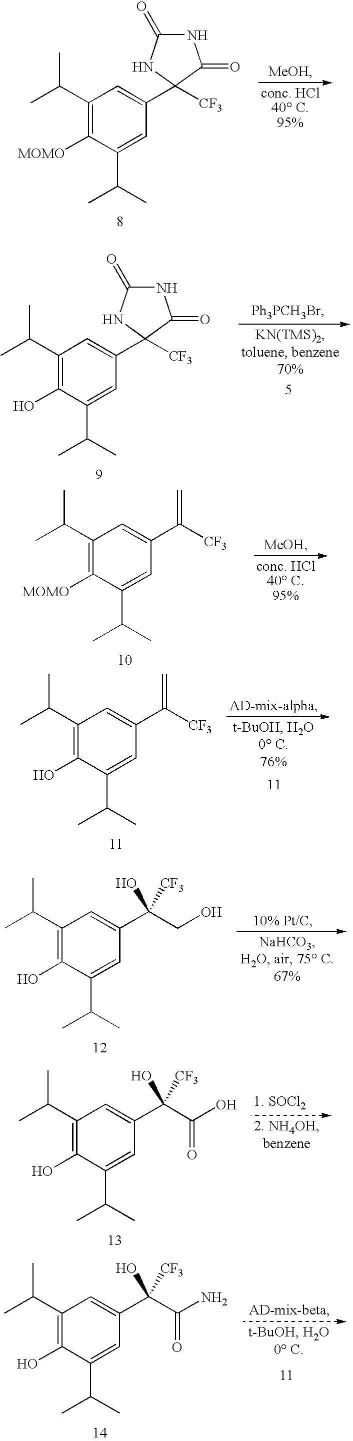 [chemical structure]
